(19) United States
(12) Patent Application Publication
(10) Pub. No.: US 2019/0175010 A1
(43) Pub. Date: Jun. 13, 2019

(54) MULTI-FUNCTION CANNULATED SURGICAL DEVICE AND SYSTEMS THEREOF

(71) Applicants: A. Jamie Riley, St. Louis, MO (US); Gary J. Schmidt, St. Louis, MO (US)

(72) Inventors: A. Jamie Riley, St. Louis, MO (US); Gary J. Schmidt, St. Louis, MO (US)

(21) Appl. No.: 16/213,510

(22) Filed: Dec. 7, 2018

Related U.S. Application Data
(60) Provisional application No. 62/595,662, filed on Dec. 7, 2017.

(51) Int. Cl. A61B 1/313; A61B 17/00; A61B 17/34

(52) U.S. Cl. CPC A61B 1/313; A61B 17/00234; A61B 1/00154; A61B 2017/3447; A61B 17/3421

(57) ABSTRACT

A surgical instrument includes a first cannula having a first pair of sidewalls and a first end wall extending along the first pair of sidewalls. The first pair of sidewalls and the first end wall at least partially define a first cavity. The surgical instrument also includes a second cannula comprising a second pair of sidewalls and a second end wall extending along the second pair of sidewalls. The second pair of sidewalls and the second end wall at least partially define a second cavity. The first pair of sidewalls are configured to slidably couple to the second pair of sidewalls such that as the first cannula moves axially along the second cannula, the first end wall moves away from the second end wall.

### BACKGROUND

The field of disclosure relates generally to the field of surgical tools, for example, endoscopic surgical tools and, more particularly, to a multi-function cannulated surgical device suitable for endoscopic applications, among others.

In traditional surgical procedures involving endoscopy, at least two incisions are typically required. A first incision allows for the insertion of a scope while a second incision accommodates a surgical tool. The traditional methodology requires significant manual dexterity as the operating physician must generally operate both the scope and surgical tool simultaneously.

In addition, in many endoscopic applications, the nature of the procedure and the treatment area results in very awkward positioning of the scope and surgical tool. For example, in the case of endoscopic plantar fasciotomy, the standard procedure requires insertion of the endoscope from one side of the patient's foot, while a cutting blade or other surgical tool is inserted from the opposite side of the foot. This orientation requires a difficult mental correction by the surgeon with respect to the direction of required movement of the surgical tool as the endoscopic is providing a view that is opposite to the orientation of the surgical tool.

Therefore, it would be advantageous to provide a multi-function surgical device that allows for the introduction of multiple types of scopes and surgical tools from the same orientation without interfering with the physician's view of the treatment area and requiring a single incision.

### SUMMARY

A surgical instrument includes a first cannula having a first pair of sidewalls and a first end wall extending along the first pair of sidewalls. The first pair of sidewalls and the first end wall at least partially define a first cavity. The surgical instrument also includes a second cannula comprising a second pair of sidewalls and a second end wall extending along the second pair of sidewalls. The second pair of sidewalls and the second end wall at least partially define a second cavity. The first pair of sidewalls are configured to slidably couple to the second pair of sidewalls such that as the first cannula moves axially along the second cannula, the first end wall moves away from the second end wall.

A surgical instrument includes a first cannula having a first interior space and a second cannula having a second interior space. The first interior space and the second interior space form a combined interior space having a first size when the first and second cannulas are in a first relative position and a second, enlarged size when the first and second cannulas are in a second relative position.

A surgical procedure includes making a single incision at a desired location and inserting a first cannula into the incision, wherein the first cannula includes a first end wall that at least partially defines a first cavity. The procedure also includes coupling a second cannula to the first cannula, wherein the second cannula includes a second end wall that at least partially defines a second cavity. The first cavity and the second cavity define a combined interior space. The procedure also includes sliding the second cannula along the first cannula from a first relative position to a second relative position such that as the first cannula moves axially along the second cannula, the first end wall moves away from the second end wall. The combined interior space has a first size in the first relative position and second, enlarged size in the second relative position.

### BRIEF DESCRIPTION OF THE DRAWINGS

FIG. 1 is a perspective view of components of a surgical system;

FIG. 2A is a perspective view of a cannulated surgical device of the surgical system shown in FIG. 1 in a first position illustrating a first cannula and a second cannula.

FIG. 2B is a cross-sectional view taken along line 2-2 in FIG. 2A.

FIG. 3 is a bottom view of the cannulated surgical device shown in FIG. 2 in an intermediate position.

FIG. 4A is a perspective view of the cannulated surgical device shown in FIG. 2 in a second position.

FIG. 4B is a cross-sectional view taken along line B-B in FIG. 4A.

FIG. 4C is a cross-sectional view taken along line C-C in FIG. 4A.

FIG. 5 is a top view of the first cannula.

FIG. 6 is a perspective view of the first cannula.

FIG. 7 is a perspective view of the second cannula.

FIG. 8 is a top view of the second cannula.

FIG. 9 is a perspective view of the cannulated surgical device assembled with a cutting insert and a scope.

FIG. 10 is an exploded top view of the cannulated surgical device and the cutting insert with the scope.

FIG. 11 is an enlarged view of a portion of the cannulated surgical device assembled with the cutting insert and the scope as shown in FIG. 9.

FIG. 12 is a top view of the cannulated surgical device assembled with a delivery mechanism and a scope.

FIG. 13 is an exploded perspective view of the cannulated surgical device and the delivery mechanism with the scope.

FIG. 14 is a bottom view of the delivery mechanism shown in FIGS. 12 and 13.

FIG. 15 is an enlarged view of a portion of the cannulated surgical device assembled with the delivery mechanism and the scope as shown in FIG. 9.

FIG. 16 is a top view of the second cannula assembled with an obturator.

FIG. 17 is a side view of the obturator shown in FIG. 16.

### DETAILED DESCRIPTION

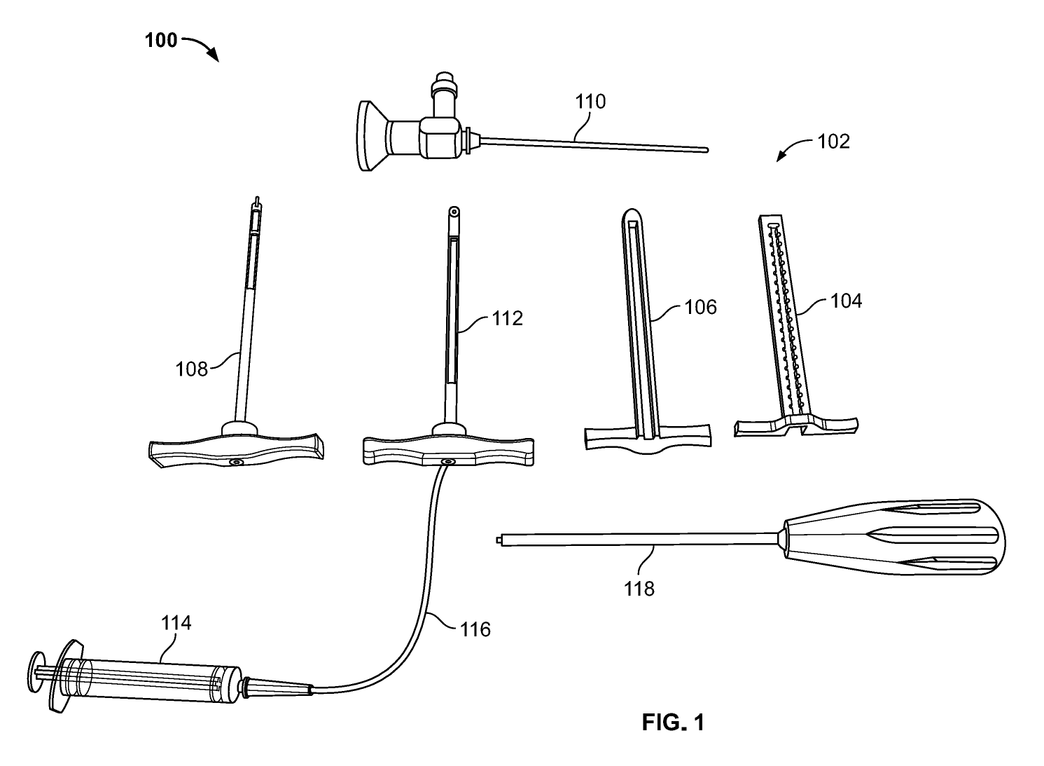

FIG. 1 is a perspective view of components of a surgical system 100. System 100 includes a cannulated surgical device 102 including a first cannula 104 and a second cannula 106. In the exemplary embodiment, second cannula 106 is inserted into an incision and first cannula 104 slides along second cannula 106 to widen the incision and to form a cavity between cannulas 104 and 106 that is configured to receive other components of system 100 as described herein. System 100 also includes a cutting mechanism 108 and a probe 110 that are simultaneously inserted into the cavity defined by the cannulas 104 and 106 to enable the physician to view what needs to be cut from within the cutting mechanism 108, as described herein. System 100 also includes a delivery mechanism 112 for delivering a biologic or medicine into the patient's body. A source of the fluid, for example a syringe 114, is coupled to delivery mechanism 112 via a tube 116. Delivery mechanism 112 is inserted into the cavity defined by cannulas 104 and 106 to facilitate delivering the fluid within the incision. Additionally, system 100 includes an obturator 118 that couples to and slides along second cannula 106 before obturator 118 and second cannula 106 are inserted into the incision. Obturator 118 provides a gripping mechanism to assist the physician with inserting second cannula into the incision.

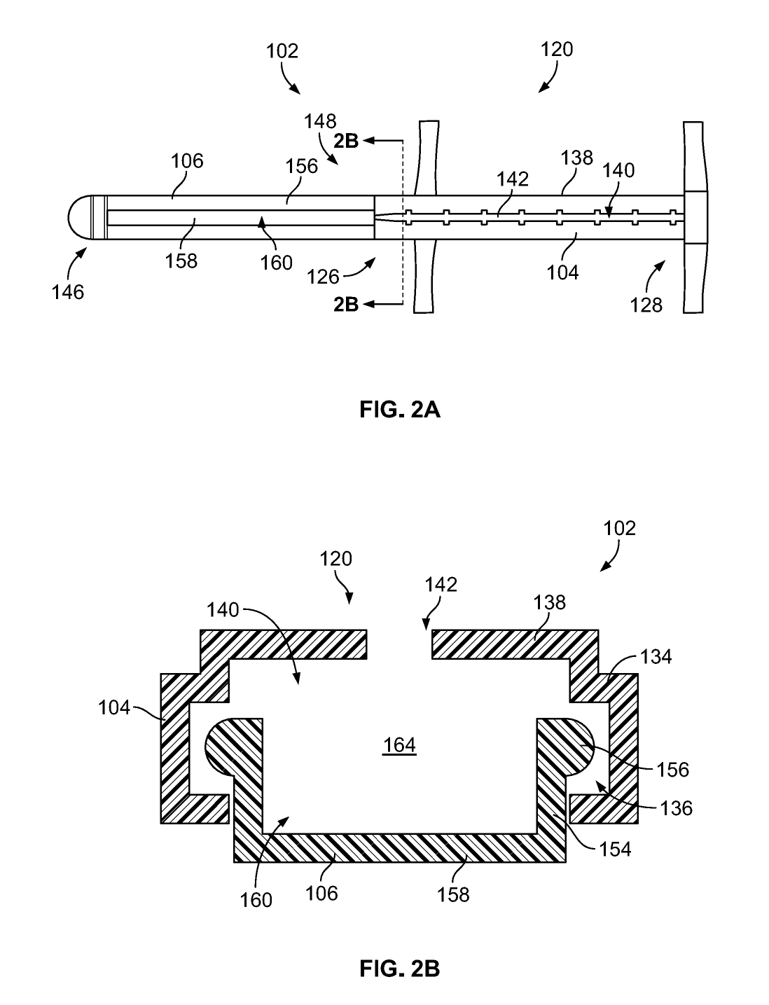

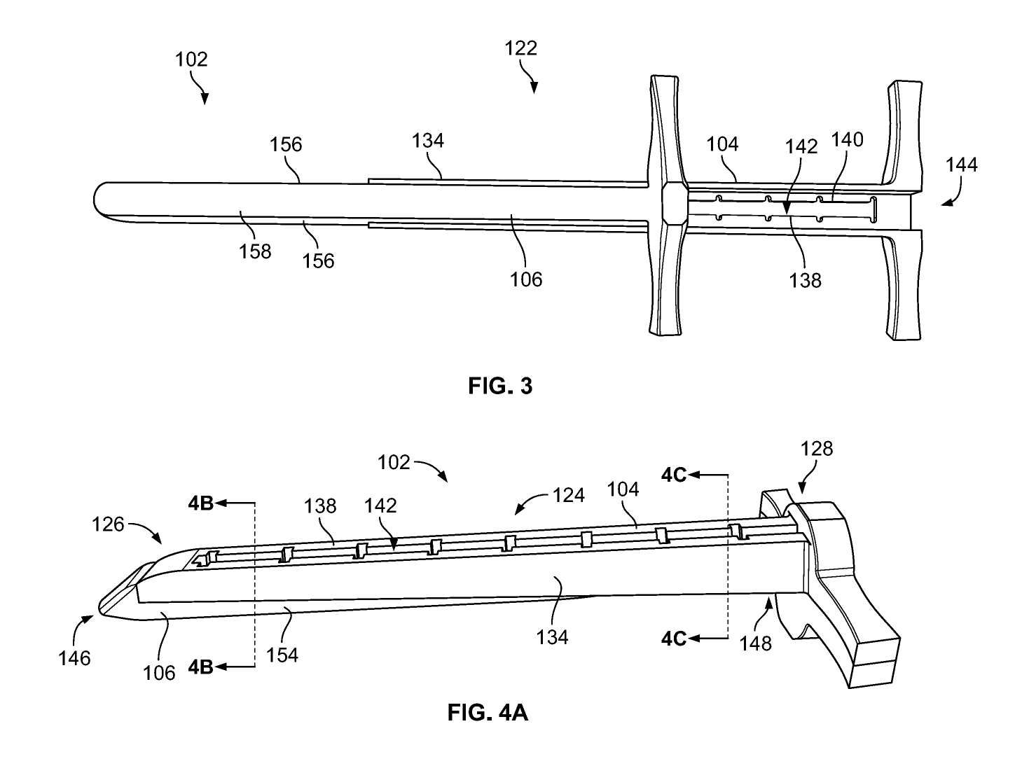

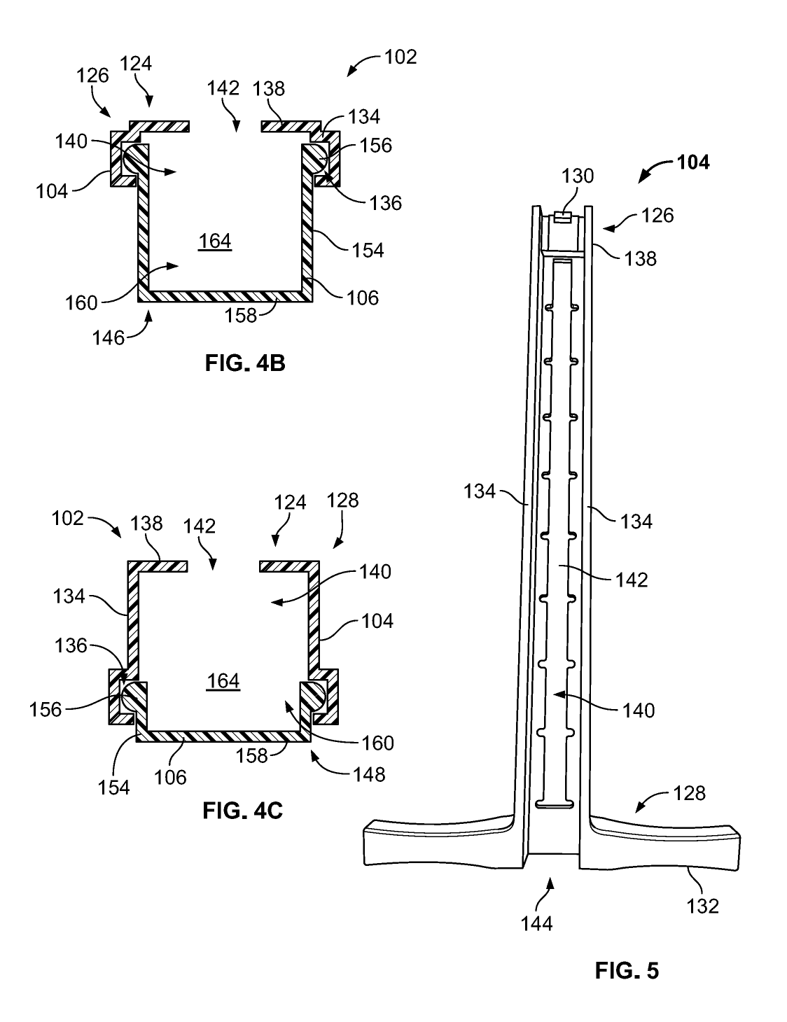

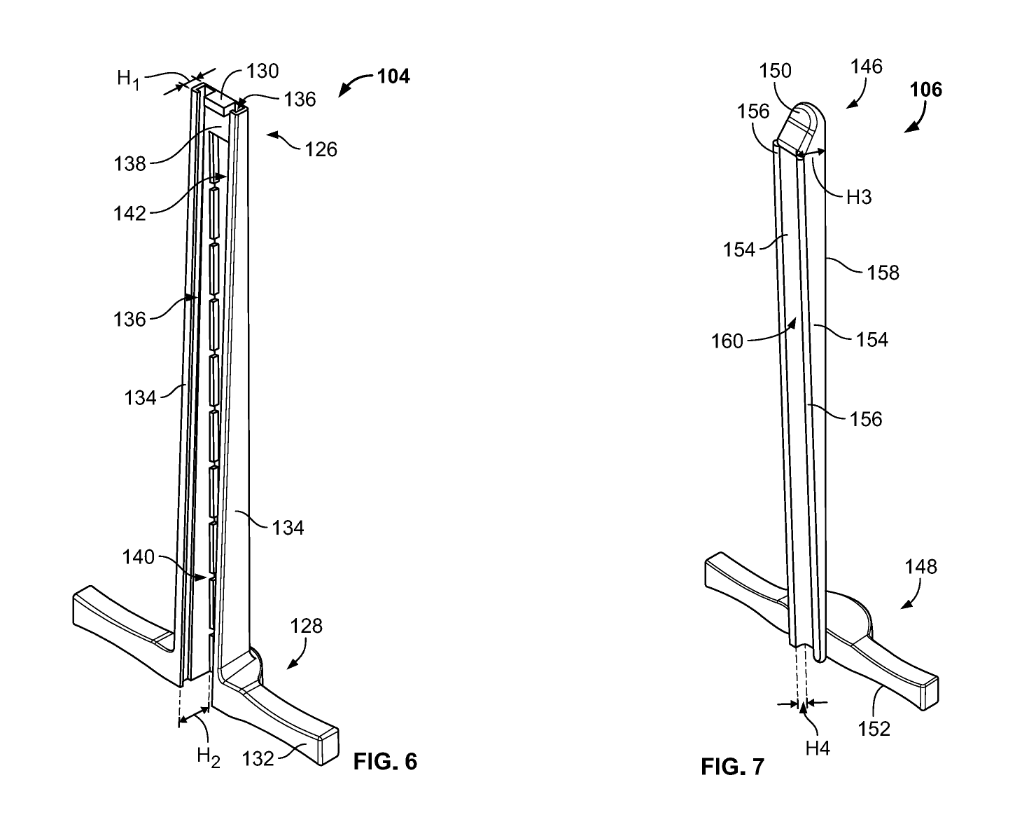

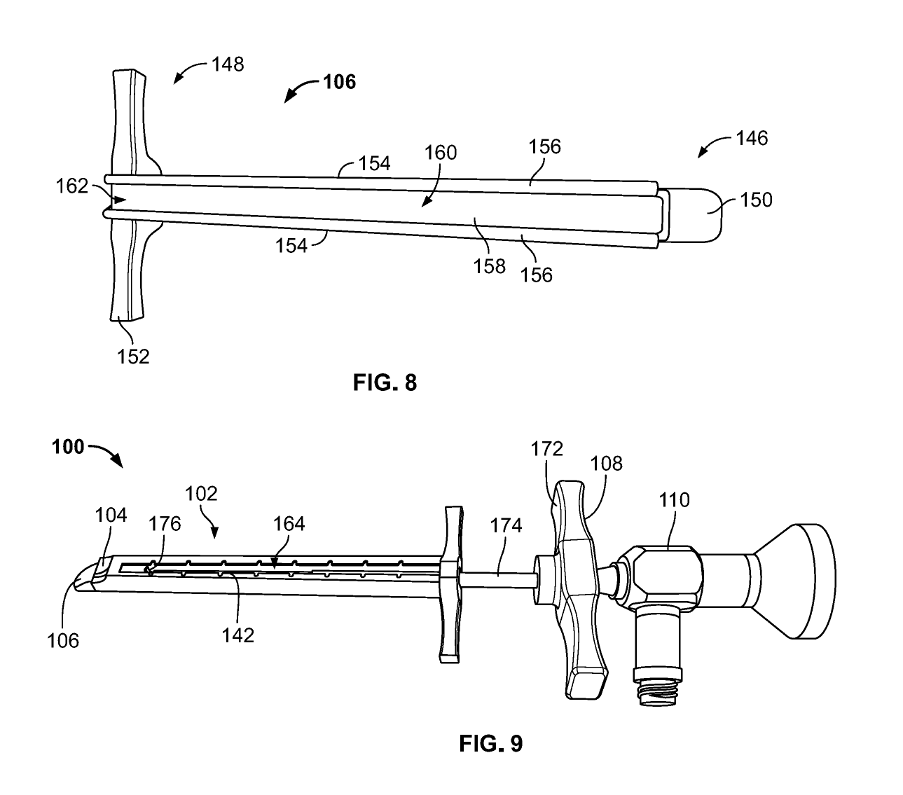

FIG. 2 is a perspective view of a cannulated surgical device 102 of surgical system 100 shown in FIG. 1 in a first position 120. FIG. 3 is a top perspective view of cannulated surgical device 102 in an intermediate position 122. FIG. 4 is a bottom view of cannulated surgical device 102 in a second position 124. FIG. 5 is a top view of first cannula 104. FIG. 6 is a perspective view of first cannula 104. FIG. 7 is a perspective view of second cannula 106. FIG. 8 is a top view of second cannula 106.

In the exemplary embodiment, as best shown in FIGS. 5 and 6, first cannula 104 includes a first, distal end 126 and a second, proximal end 128. First end 126 includes a stop 130 configured to contact a tip of second cannula 106, as described in further detail below, and second end 128 includes a handle 132 to facilitate use of first cannula 104. First cannula 104 also includes a pair of sidewalls 134 that extend from first end 126 to second end 128. In the exemplary embodiment, sidewalls 134 are parallel to each other and are substantially planar along their lengths. Alternatively, sidewalls 134 may be non-parallel and may be any shape that facilitates operation of cannulated surgical device 102 as described herein. Additionally, sidewalls 134 include a tapered height along their lengths. More specifically, sidewalls 134 include a first height H1 proximate first end 126 and a second height H2 proximate second end 128. In the exemplary embodiment, first height H1 is less than second height H2 such that the height of sidewalls 134 increases from first end 126 to second end 128. Alternatively, the opposite may be true where first and second heights H1 and H2 are switched and the height of sidewalls 134 decreases from first end 126 to second end 128.

Each sidewall 134 also includes a groove 136 defined therein that extends the length of sidewall 134 from first end 126 to second end 128. In the exemplary embodiment, grooves 136 are configured to receive corresponding guides on second cannula 106 to enable first cannula 104 to slidably engage with and move along second cannula 106, as described herein. The height of grooves 136 is constant between ends 126 and 128 such that, at first end 126, the first height H1 of sidewalls 134 is substantially similar to the height of grooves 136. Whereas, at second end 128, the second height H2 of sidewalls 134 is larger than the height of grooves 136.

In the exemplary embodiment, first cannula 104 also includes a first end wall, or top wall 138 that extends between sidewalls 134 the length of first cannula 104 from first end 126 to second end 128. Together, top wall 138 and sidewalls 134 form a U-shape that defines a first cavity 140 within an interior of the U-shaped first cannula 104. Alternatively, walls 138 and 134 define any shape that defines first cavity 140 within first cannula 104. Because first cavity 140 is partially defined by tapered sidewalls 134, first cavity 140 is also tapered along its length. More specifically, first cavity 140 includes a first cross-sectional area defined by sidewalls 134 at first height H1 proximate first end 126. Similarly, first cavity 140 includes a second cross-sectional area defined by sidewalls 134 at second height H2 proximate second end 128. In the exemplary embodiment, the first cross-sectional area of first cavity at first end 126 is smaller than the second cross-sectional area of first cavity 140 at second end 128 because the height of sidewalls 134 is smaller at first end 126 than at second end 128. Alternatively, in embodiments where the height of sidewalls 134 is larger at first end 126 than at second end 128, the cross-sectional areas of first cavity 140 are correspondingly switched such that the first area is larger at first end 126 than at second end 128.

Top wall 138 also includes an elongated first opening 142 that extends a majority of the length of top wall 138 between first end 126 and second end 128. As described herein, first opening 142 enables tools or fluids in first cavity 140 to extend radially from the interior of first cannula 104, through first opening 142 in top wall 138, and into an exterior of first cannula 104 to contact the patient's body. Furthermore, a second opening 144 is defined in first cannula 104 proximate second end 128 at handle 132. Second opening 144 enables the physician to axially insert a tool into first cavity 140 from second end 128 of first cannula 104.

As best shown in FIGS. 7 and 8, in the exemplary embodiment, second cannula 106 includes a first, distal end 146 and a second, proximal end 148. First end 146 includes a tapered tip 150 configured to facilitate insertion of second cannula 106 into an incision, as described in further detail below. Alternatively, first end 146 includes a variety of different tips that may be selected based upon the procedure in which cannulated surgical device 102 is being used. For example, second cannula 106 may include a split, beveled, or a spatula tip.

Second end 148 also includes a handle 152 to facilitate use of second cannula 106. Second cannula 106 also includes a pair of sidewalls 154 that extend from first end 146 to second end 148. In the exemplary embodiment, sidewalls 154 are parallel to each other and are substantially planar along their lengths. Alternatively, sidewalls 154 may be non-parallel and may be any shape that facilitates operation of cannulated surgical device 102 as described herein. Additionally, sidewalls 154 include a tapered height along their lengths. More specifically, sidewalls 154 include a third height H3 proximate first end 146 and a fourth height H4 proximate second end 148. In the exemplary embodiment, third height H3 is greater than fourth height H4 such that the height of sidewalls 154 decreases from first end 146 to second end 148. Alternatively, the opposite may be true where third and fourth heights H3 and H4 are switched and the height of sidewalls 154 increases from first end 146 to second end 148. More specifically, when the height of sidewalls 134 of first cannula 104 increases from first end 126 to second end 128, then the height of sidewalls 154 of second cannula 106 decreases from first end 146 to second end 148, and vis versa.

Each sidewall 154 also includes a guide rail 156 extending therefrom in a direction away from the opposite sidewall 154. Guides 156 extend the length of sidewalls 134 from first end 126 to second end 128. In the exemplary embodiment, guides 156 are configured to engage corresponding grooves 136 on first cannula 104 to enable first cannula 104 to slidably engage with and move along second cannula 106, as described herein. The height of guides 156 is constant between ends 126 and 128 to correspond with grooves 136.

In the exemplary embodiment, second cannula 106 also includes a second end wall, or bottom wall 158 that extends between sidewalls 154 the length of second cannula 106 from first end 146 to second end 148. Together, bottom wall 158 and sidewalls 154 form a U-shape that defines a second cavity 160 within an interior of the U-shaped second cannula 106. Alternatively, walls 158 and 154 define any shape that defines second cavity 160 within second cannula 106. Because second cavity 160 is partially defined by tapered sidewalls 154, second cavity 160 is also tapered along its length. More specifically, second cavity 160 includes a third cross-sectional area defined by sidewalls 154 at third height H3 proximate first end 146. Similarly, second cavity 160 includes a fourth cross-sectional area defined by sidewalls 154 at fourth height H4 proximate second end 148. In the exemplary embodiment, the third cross-sectional area of second cavity 160 at first end 146 is greater than the fourth cross-sectional area of second cavity 160 at second end 148 because the height of sidewalls 154 is greater than at first end 146 than at second end 148. Alternatively, in embodiments where the height of sidewalls 154 is smaller at first end 146 than at second end 148, the cross-sectional areas of second cavity 160 are correspondingly switched such that the third area is smaller at first end 146 than at second end 148.

Bottom wall 158 is substantially solid and continuous along its length and between sidewalls 154. Additionally, bottom wall 158 also at least partially defines an opening 162 in second cannula 106 proximate second end 148 at handle 152. Opening 162 enables the physician to axially insert a tool into second cavity 160 from second end 148 of second cannula 106.

As described above, FIG. 2A is a perspective view of a cannulated surgical device 102 of surgical system 100 shown in FIG. 1 in a first position 120. FIG. 2B is a cross-sectional view taken along line 2-2 in FIG. 2A. FIG. 3 is a bottom view of cannulated surgical device 102 in an intermediate position 122. FIG. 4A is a perspective view of cannulated surgical device 102 in a second position 124. FIG. 4B is a cross-sectional view taken along line B-B in FIG. 4A. FIG. 4C is a cross-sectional view taken along line C-C in FIG. 4A.

In operation, such as during a surgical procedure, a single incision is made at a desired location on the patient. Second cannula 106 is then inserted into the incision. First cannula 104 is slidably coupled to second cannula 106 via guides 156 of second cannula 106 engaging grooves 136 of first cannula 104. When cannulas 104 and 106 are coupled together, sidewalls 134 and 152 are parallel to each other and top wall 138 is parallel to bottom wall 158 such that cannulas 104 and 106 form cannulated surgical device 102 having an interior cavity 164. More specifically, first cavity 140 of first cannula 104 and second cavity 160 of second cannula 106 combine, and may partially overlap to form interior cavity 164 of device 102. In the first position 120, cavity 164 includes a first cross sectional area that is relatively small because the portions of cannulas 104 and 106 that overlap to form cavity 164 (first end 126 and second end 148) are the portions where sidewalls 134 and 154 are shortest.

The physician then slides first cannula 104 along second cannula 106 from first position 120, through the intermediate position 122, and until first cannula reaches the second position 124. As shown in FIGS. 4B and 4C, interior cavity 164 has a substantially similar cross-sectional area proximate both first ends 126 and 146 and second ends 128 and 148 when cannulas 102 and 104 are in the second position 124. The cross-sectional area of interior cavity 164 in second position 124 at any location along cavity 164 is larger than the cross-sectional area of interior cavity 164 in the first position 120. As such, as first cannula 104 moves axially along second cannula 106, first cannula top wall 138 moves radially away from second cannula bottom wall 158, and cavity 164 defined between first cannula 104 and second cannula 106 enlarges as first cannula 104 moves axially along second cannula 106.

In the second position 124, sidewalls 134 of first cannula 104 lies substantially against the exterior surfaces of sidewalls 154 of second cannula 106. This position minimizes the total exterior thickness of the device 102, thereby easing insertion and extraction of the device 102 through an incision. In the expanded position 124, the total, combined, interior cavity 164 of the device 104 is maximized to allow for the introduction of multiple instruments into interior cavity 164. This allows both a scope and another surgical instrument to be used by the physician at the same time.

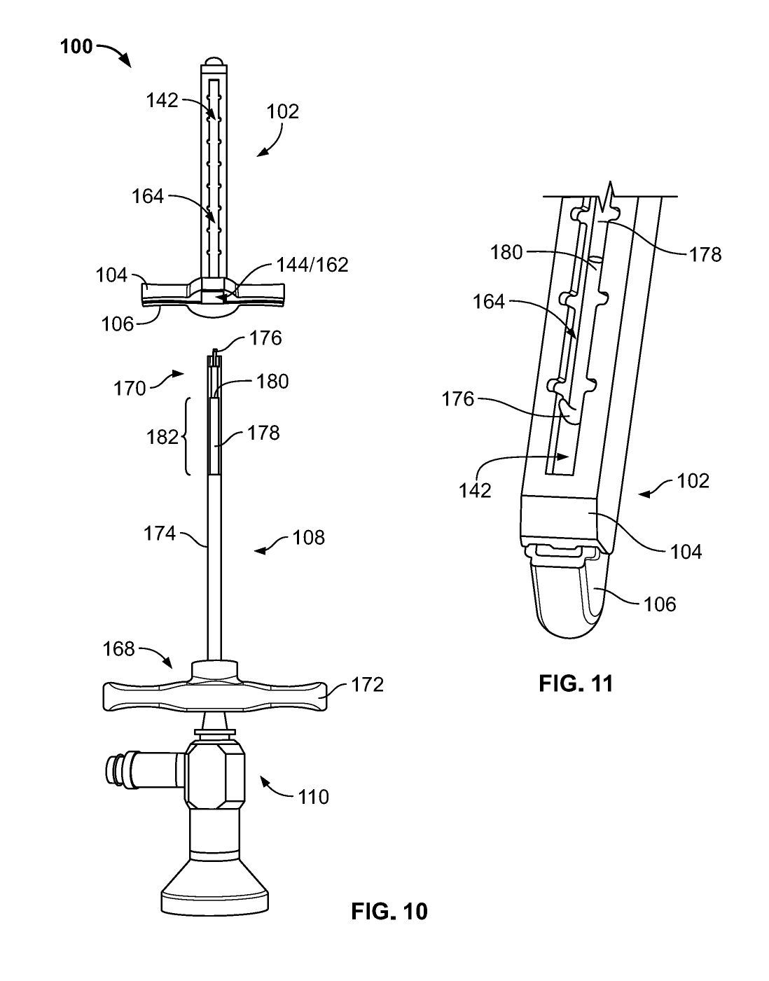

FIG. 9 is a perspective view of the cannulated surgical device 102 assembled with cutting mechanism 108 and scope 110. FIG. 10 is an exploded top view of the cannulated surgical device 102 and the cutting mechanism 108 with scope 110. FIG. 11 is an enlarged view of a portion of the cannulated surgical device 102 assembled with the cutting mechanism 108 with the scope 110. In the exemplary embodiment, cutting mechanism 108 including a first end 168, a second end 170, a handle 172 at first end 168, a hollow shaft 174 extending between ends 168 and 170, and a blade 176 coupled to shaft 174 proximate first end 168.

The cutting mechanism 108 may also be provided with one or more surgical tools that are selected for a particular procedure and are known within the field of endoscopic surgery. In the case of the illustrated embodiment, a cutting mechanism 108 suitable for endoscopic plantar fasciotomy (“EPF”), or similar cutting applications, is shown. This form of the cutting mechanism 108 is provided with a cutting blade 176 at the second end 170. This particular version has a hooked blade specific to EPF and designed to cut bands of the plantar fascia. This particular version may also have applicability in carpal tunnel release and gastric resection procedures, among others.

Although described herein as a cutting mechanism with a hooked blade, it should be noted that this is a non-limiting example of the possible variations of the mechanism 108. Variations of the mechanism 108 can be provided with different probes, curettes, grabbers, biters, biopsy tools, cauterization tips, punches, needles, and drills, and all of these variations are considered to be within the scope of the present invention. The possible attachments to the mechanism 108 include devices that serve as electrocautery probes for removal of unwanted or harmful tissue, coagulation of bleeding tissue, and sealing blood vessels to help reduce or stop bleeding. The electrocautery probe function can be incorporated into the tips of the previous listed instruments, e.g., hook, grabber, needle, drill, etc.

In the exemplary embodiment, probe 110 includes a shaft 178 that is inserted into shaft 174 of mechanism 108 until probe shaft 178 contacts a shaft stopper 180 of mechanism 108. Probe 110 may include a camera and/or a light source to enable the physician to view what needs to be cut from within the cutting mechanism 108. More specifically, mechanism shaft 174 includes a viewport 182 defined therein that enables a camera of probe 110 to “see” blade 176.

In operation, probe 110 is inserted into cutting mechanism 108 and cutting mechanism 108 with probe 110 are simultaneously inserted into cannulated surgical device 102. More specifically, cutting mechanism 108 with probe 110 are simultaneously inserted through openings 144 and 162 in second ends 128 and 148 of cannulas 104 and 106 and into interior cavity 164 of device 102. Opening 142 in top wall 138 of first cannula 104 allows for communication between the exterior and interior of cannulated surgical device 102. More specifically, opening 142 allows for surgical instruments, such as blade 176, inserted into cavity 164 of cannulated surgical device 102 to interact with the environment exterior to cannulated surgical device 102 and within the treatment area. The location, shape, and size of the opening 142 will be variable depending upon the nature of the procedure for which cannulated surgical device 102 will be used. As a non-limiting example, the opening 142 referenced above takes the form of an open slot in top wall 138 of first cannula 104. As such, the physician is able to insert the cutting mechanism in the same orientation as the cannulated surgical device 102 without interfering with the physician's view of the treatment area and requiring a single incision.

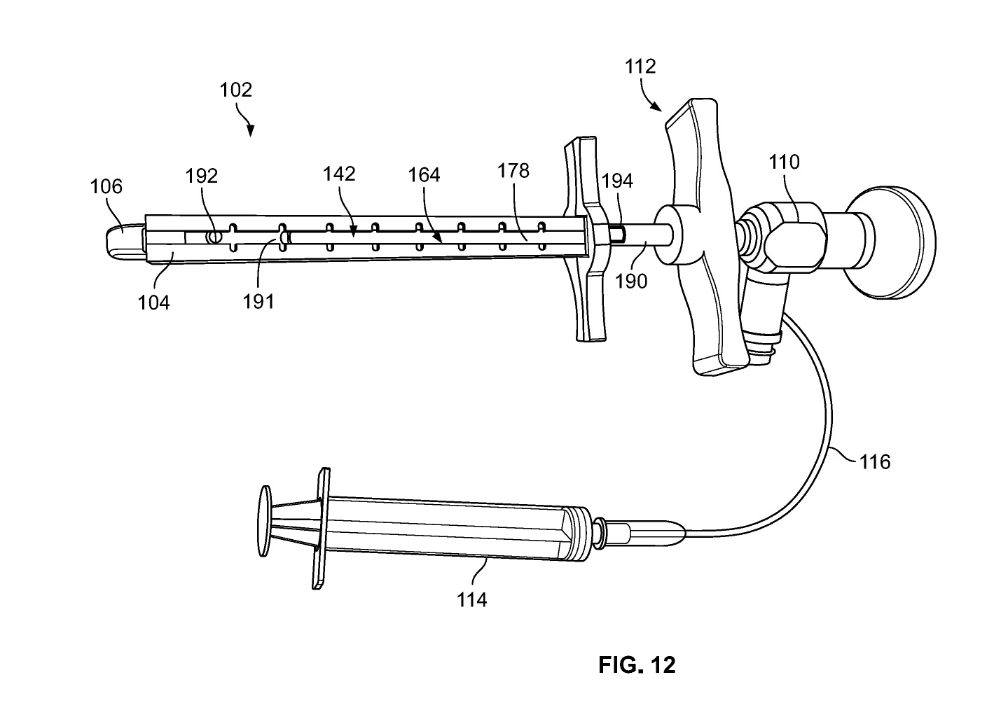

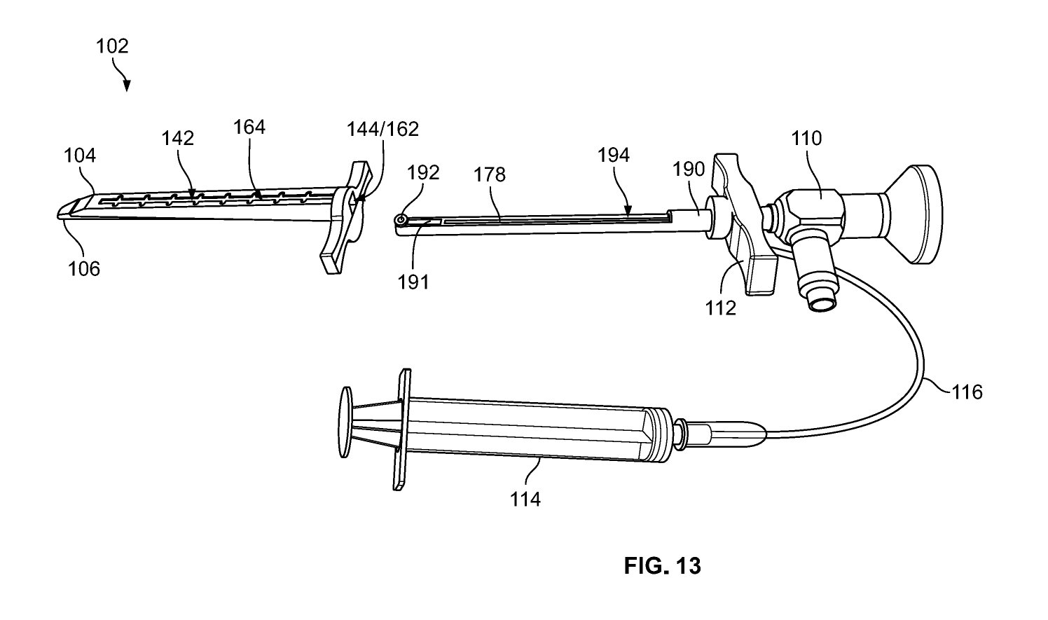

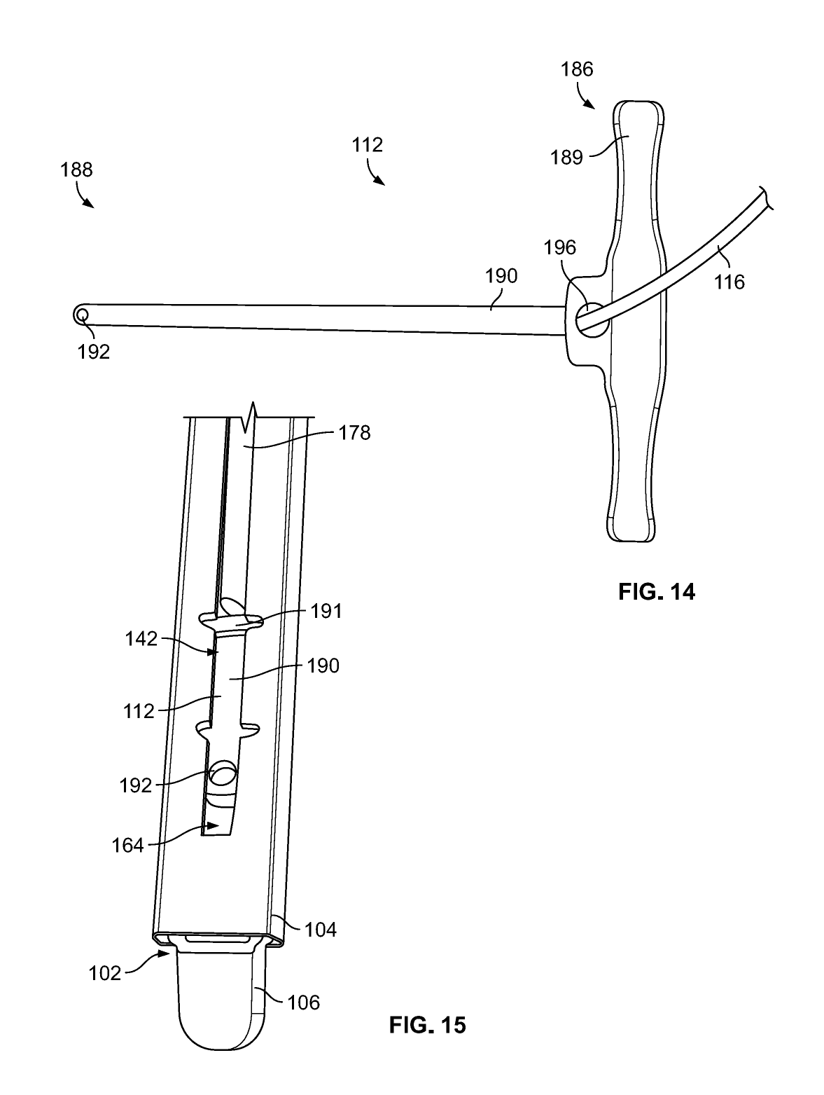

FIG. 12 is a top view of the cannulated surgical device 102 assembled with delivery mechanism 112 and scope 110. FIG. 13 is an exploded perspective view of the cannulated surgical device 102 and the delivery mechanism 112 with the scope 110. FIG. 14 is a bottom view of the delivery mechanism 112 shown in FIGS. 12 and 13. FIG. 15 is an enlarged view of a portion of the cannulated surgical device 102 assembled with the delivery mechanism 112 and the scope 110 as shown in FIG. 9.

In the exemplary embodiment, delivery mechanism 112 includes a first end 188, a second end 186, a handle 189 at second end 186, and a hollow shaft 190 extending between ends 188 and 186. Shaft 190 includes a probe stop 191 and a nozzle 192 positioned at first end 188. A viewport 194 is defined in shaft 190 to enable the camera in probe 110 to “see” the area. Tube 116 is inserted into an opening 196 proximate second end 186 and extends the length of shaft 190, beneath and parallel to probe shaft 178, and is coupled to nozzle 192.

In operation, probe 110 is inserted into delivery mechanism 112 and delivery mechanism 112 with probe 110 are simultaneously inserted into cannulated surgical device 102. More specifically, delivery mechanism 112 with probe 110 are simultaneously inserted through openings 144 and 162 in second ends 128 and 148 of cannulas 104 and 106 and into interior cavity 164 of device 102. Opening 142 in top wall 138 of first cannula 104 allows for communication between the exterior and interior of cannulated surgical device 102. More specifically, opening 142 allows for the fluid within syringe 114 to flow through tube 116 and be discharged through nozzle 192 into cavity 164 of cannulated surgical device 102 and through opening 142 to interact with the environment exterior to cannulated surgical device 102 and within the treatment area. As such, the physician is able to insert the delivery mechanism in the same orientation as the cannulated surgical device 102 without interfering with the physician's view of the treatment area and requiring a single incision.

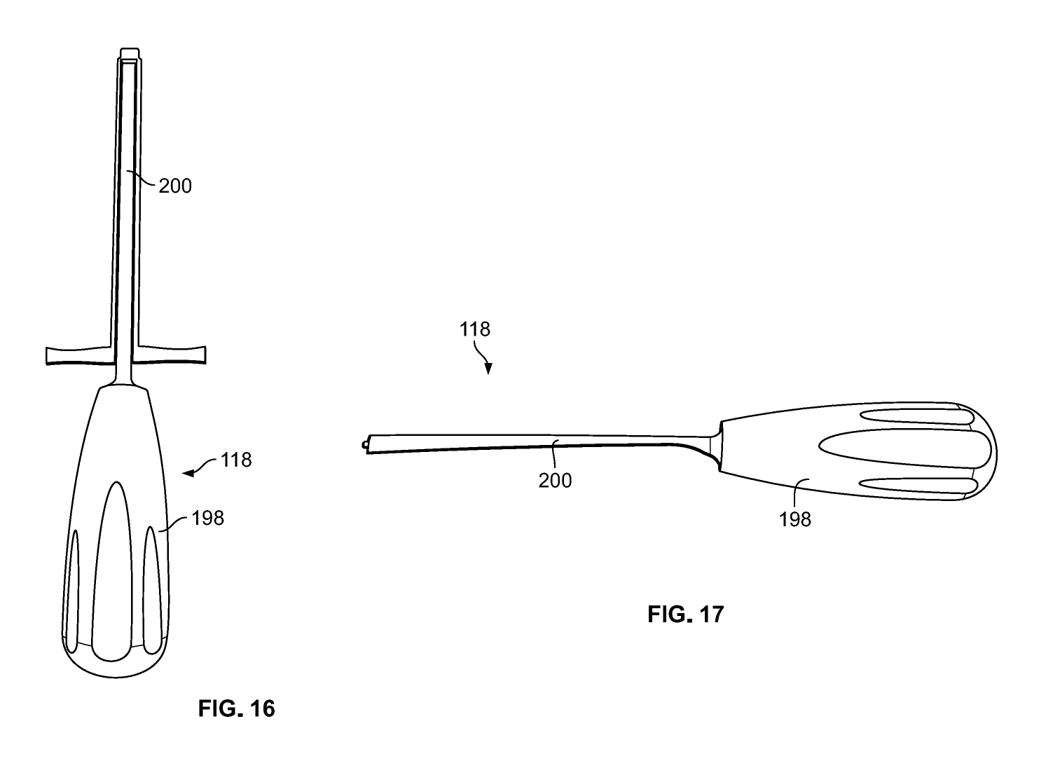

FIG. 16 is a top view of the second cannula 106 assembled with an obturator 118, and FIG. 17 is a side view of the obturator 118. In the exemplary embodiment, obturator 118 includes a handle portion 198 and a shaft portion 200. Shaft portion 200 is tapered similar to sidewalls 134 of first cannula 104 such that obturator 118 is slidably couplable with second cannula 106. More specifically, once the incision is made, the obturator 118 is then coupled to second cannula 106 and together the obturator 118 and second cannula 106 are inserted into the incision. Handle portion 198 facilitates gripping obturator 118 to assist with inserting second cannula 106 into the incision.

As a non-limiting example of use of the illustrated system 100, the steps involved in using the device in an EPF procedure are provided. The physician first makes a 1 cm incision on the medial aspect of the patient's foot, at or just proximal to the high point of the arch of the foot. Blunt dissection lateral to the middle band of the plantar fascia using the cannulated member 202 then occurs. Next, the obturator 118 is coupled to second cannula 106 and inserted into the incision. Then obturator 118 is removed and first cannula 104 is slidably coupled to second cannula 106 within the incision to form cavity 164 within device 102. Next, mechanism 108 is fitted with probe 110, for example an endoscope, e.g. a 2.7 mm/70°/4″ scope. The mechanism 108 with the inserted scope 110 is introduced into the cannulated device 102 to allow for viewing of the plantar fascia and simultaneous cutting. When the mechanism 108 with the inserted scope 110 is in the proper position, mechanism 108 is raised to extend blade 176 through opening 142 in first cannula 104 to cut the fascia as required. When cutting is complete, mechanism 108 is dropped back down into cavity 164 and retracted from the cannulated device 102 with probe. The cannulated device 102 is then removed through the incision.

In general, embodiments of the present invention are particularly well-suited for a variety of applications, including, for example, endoscopic procedures such as fascial release—plantar, carpal, ulnar, etc.—muscle release—gastric/soleous, etc. Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose various embodiments, which include the best mode, to enable any person skilled in the art to practice those embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A surgical instrument comprising:
a first cannula comprising a first pair of sidewalls and a first end wall extending along said first pair of sidewalls, wherein said first pair of sidewalls and said first end wall at least partially define a first cavity; and
a second cannula comprising a second pair of sidewalls and a second end wall extending along said second pair of sidewalls, wherein said second pair of sidewalls and said second end wall at least partially define a second cavity;
wherein said first pair of sidewalls are configured to slidably couple to said second pair of sidewalls such that as said first cannula moves axially along said second cannula, said first end wall moves away from said second end wall.

2. The surgical instrument in accordance with claim 1, wherein said first end wall moves radially away from said second end wall.

3. The surgical instrument in accordance with claim 1, wherein said first pair of sidewalls are oriented parallel to said second pair of sidewalls.

4. The surgical instrument in accordance with claim 1, wherein said first end wall is oriented parallel to said second end wall.

5. The surgical instrument in accordance with claim 1, wherein one of said first pair of sidewalls or said second pair of sidewalls each comprise a groove extending along a length of said first pair of sidewalls or said second pair of sidewalls.

6. The surgical instrument in accordance with claim 5, wherein the remaining one of said first pair of sidewalls or said second pair of sidewalls comprises a guide rail extending a length of said first pair of sidewalls or said second pair of sidewalls, wherein said guide rails are configured to engage said grooves to facilitate slidable engagement of said first cannula with said second cannula.

7. The surgical instrument in accordance with claim 1, wherein each of said first pair of sidewalls and said second pair of sidewalls define a tapered height.

8. The surgical instrument in accordance with claim 1, wherein said first cannula comprises a proximal end where said first pair of sidewalls have a first height, and a distal end where said first pair of sidewalls have a second height different than the first height.

9. The surgical instrument in accordance with claim 8, wherein said second cannula comprises a proximal end where said second pair of sidewalls have a third height, and a distal end where said second pair of sidewalls have a fourth height different than the third height.

10. The surgical instrument in accordance with claim 9, wherein said first height is substantially similar to said fourth height, and wherein said second height is substantially similar to said third height.

11. The surgical instrument in accordance with claim 1, wherein said first cavity and said second cavity combine to define an interior space having a first size when said first cannula and said second cannula are in a first relative position, and wherein said interior space includes a second, enlarged size when said first cannula and said second cannula are in a second relative position.

12. The surgical instrument in accordance with claim 1, further comprising a cartridge mechanism configured for insertion into said first cavity and said second cavity defined between said first end wall and said second end wall, wherein said cartridge mechanism comprises and engagement device at a distal end of said cartridge mechanism, said engagement device configured to interact with an environment external to said first cannulas and said second cannula through an opening in first end wall.

13. The surgical instrument in accordance with claim 12, wherein said cartridge mechanism comprises a cutting mechanism and said engaging device comprises a blade.

14. The surgical instrument in accordance with claim 12, wherein said cartridge mechanism comprises a delivery mechanism and said engaging device comprises a nozzle.

15. The surgical instrument in accordance with claim 12, wherein said cartridge mechanism comprises a hollow shaft having a viewport portion defined therethrough, wherein said viewport is aligned with said opening.

16. The surgical instrument in accordance with claim 15, further comprising a scope comprising a shaft configured for insertion into said hollow shaft, wherein said scope comprises a camera configured to be aimed through said viewport and through said opening. wherein said cartridge mechanism comprises a delivery mechanism and said engaging device comprises a nozzle.

17. A surgical instrument comprising:
a first cannula having a first interior space; and
a second cannula having a second interior space, the first interior space and the second interior space forming a combined interior space having a first size when said first and second cannulas are in a first relative position and a second, enlarged size when said first and second cannulas are in a second relative position.

18. The surgical instrument in accordance with claim 17, wherein said first cannula moves away from said second cannula as said first cannula moves axially along said second cannula from said first relative position to said second relative position.

19. The surgical instrument in accordance with claim 17, wherein said first cannula comprises a first end wall at least partially defining the first interior space and said second cannula comprises a second end wall at least partially defining the second interior space, said first and second cannulas are configured such that said first end wall moves away from said second end wall as said first cannula moves axially along said second cannula from said first relative position to said second relative position.

20. A surgical procedure comprising:
making a single incision at a desired location;
inserting a first cannula into the incision, wherein the first cannula includes a first end wall that at least partially defines a first cavity;
coupling a second cannula to the first cannula, wherein the second cannula includes a second end wall that at least partially defines a second cavity, wherein the first cavity and the second cavity define a combined interior space;
sliding the second cannula along the first cannula from a first relative position to a second relative position such that as the first cannula moves axially along the second cannula, the first end wall moves away from the second end wall, and wherein the combined interior space has a first size in the first relative position and second, enlarged size in the second relative position.
